(19) United States
(12) Patent Application Publication
(10) Pub. No.: US 2026/0047954 A1
(43) Pub. Date: Feb. 19, 2026

(54) Pocket wizz urine collection device

(71) Applicant: Dominic Tucker, Inglewood, CA (US)

(72) Inventor: Dominic Tucker, Inglewood, CA (US)

(21) Appl. No.: 18/808,785

(22) Filed: Aug. 19, 2024

(51) Int. Cl. A61F 5/453; A61L 9/013

(52) U.S. Cl. CPC A61F 5/453; A61L 9/013

(57) ABSTRACT

The present invention relates to a portable bag housed in a box like structure for collecting urine, wherein the bag is housed in a compact condition inside a box which comprises a front, back, bottom and at least two side edges, with a bag opening formed at the opposite end of the bottom. The present invention is a handheld disposable and composable urinal for male persons for collecting and solidifying waste.

### BACKGROUND OF THE INVENTION

### Field of the Invention

The present invention relates to urine collection systems and more particular to a portable urine collection bag housed within a box container. The present invention relates to hygiene products specifically to an environmentally friendly urinal that is design to be disposable and fully compostable after use.

### Prior Art

A need exists for camping, long-term travel and the like, where it is not always possible to get to a toilet on time. Today, there is an increased focus on reducing the risk of infection in the wild by preventing people in the wild from endangering other people traveling in the area. Similarly, there is an increased focus on reducing the risk of infection among persons traveling abroad, where poor hygiene in toilet visits can lead to the rapid spread of infections and diseases.

However, the collection of urine may be done with a well-known urine flask formed of rigid plastic material or of glass, which is closed by a lid being squeezed or screwed on the opening. These urinary flasks have the disadvantage that the urine can flush around the flask in connection with the handling of the flask, with the risk that the urine may splash out of the flask and consequent odor nuisance.

A more convenient way of collecting urine can be done with a TravelJohn™ 2 tissue pouch of the brand TravelJohn™ comprising a bag with a rigid plastic collar where a superabsorbent pad which converts the urine into a gel is placed in the bag. This bag has the disadvantage that the collar cannot be compressed and where there is a risk that the collar or assembly around the collar may break during handling and storage. Furthermore, the collar forms a small elliptical shaped aperture that allows the collar to be positioned relatively precisely to avoid spills. A urine bag from the company Gleco A/S describes a similar solution, where the bag comprises two cords in the opening for closing the bag and where a super absorbent pad placed in the bag converts the urine into a gel. This bag has the disadvantage that the user himself has to keep the bag open while the user takes care as the bag is not stiffened around the opening. This results in more cumbersome handling and an increased risk of waste.

Therefore there is a need for a urine collection system which is portable easy to use and environmentally disposable. The present invention solves all these problems in a unique and novel way.

### SUMMARY OF THE INVENTION

The present invention relates to a portable bag housed in a box like structure for collecting urine, wherein the bag is housed in a compact condition inside a box which comprises a front, back, bottom and at least two side edges, with a bag opening formed at the opposite end of the bottom.

The objective of the present invention is to provide a bag for collecting urine which is easy to use and simple to hold open.

Another objective of the present invention is to provide a urine bag which can absorb the liquid in the bag.

Yet another objective of the present invention is to provide a urine bag which can be used by the user in question in a simple manner.

Still yet another object of the present invention is to provide a urine bag which can be disposed of after use in a hygienically and environmentally sound manner.

### BRIEF DESCRIPTION OF DRAWINGS

FIG. 1 is a perspective view of a box for housing a urine bag (not shown) in accordance with the present invention;

FIG. 2 is the perspective view shown in FIG. 1 showing in phantom line further illustrating an interiorly housed urine bag in accordance with the present invention;

FIG. 3 is a graphical side view of FIG. 2 in accordance with the present invention.

FIG. 4 is a perspective view of FIG. 2 illustrating the urine bag in a deployed condition.

FIG. 5 is a perspective view of FIG. 2 illustrating the urine bag in a deployed condition and in use in accordance with the present invention.

### DESCRIPTION OF THE INVENTION

The following description is presented to enable a person skilled in the art to make and use the invention and is provided in the context of a particular application and its requirements. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Various terms are used herein. To the extent a term used in a claim is not defined below, it should be given the broadest definition persons in the pertinent art have given that term as reflected in printed publications and issued patents at the time of filing.

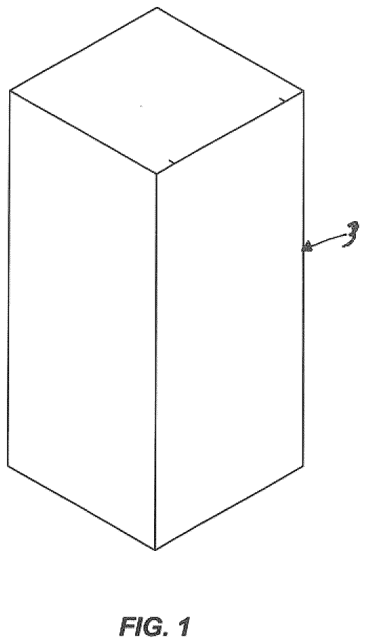

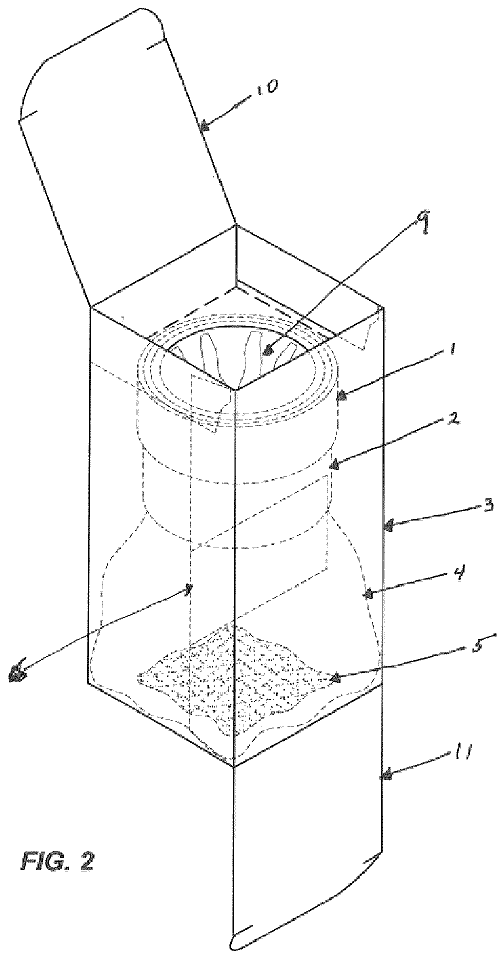

Referring now to FIGS. 1 and 2, a urine collection device, comprising: A portable bag 4 housed in a box like structure 3, wherein the bag 4 is housed in a compact condition inside the box 3, as shown in FIG. 2. Turning back to FIG. 1, the box 3 defines front, back, a bottom flap 11, a top flap 10 and at least two side edges, with a top opening 9 formed at the opposite end of the bottom of the bag 4.

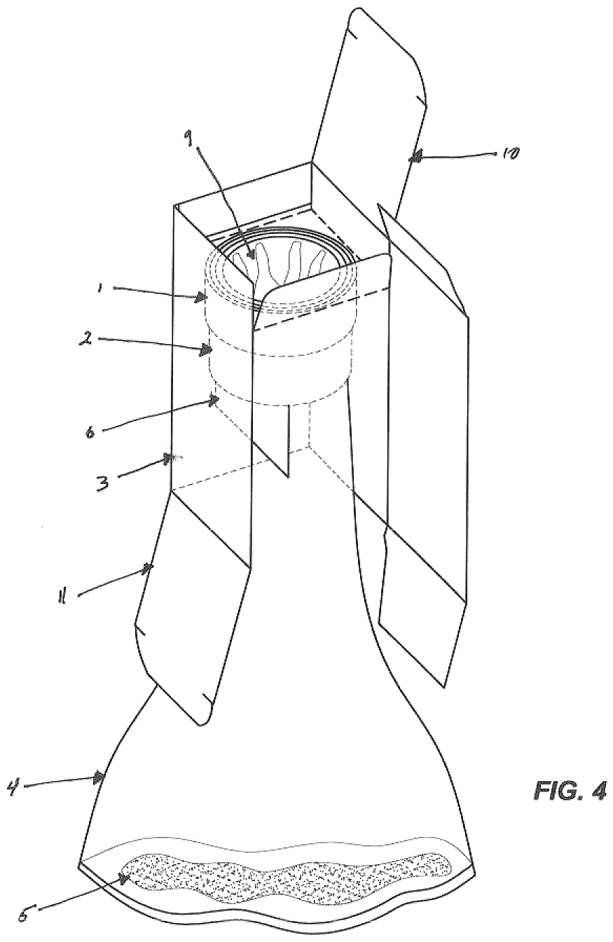

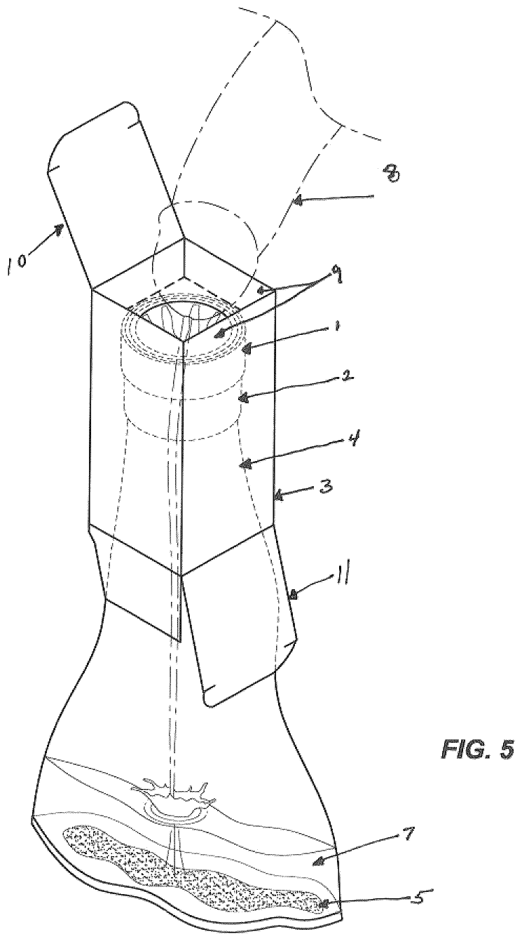

Referring now to FIGS. 1 through 5 and more specifically to the top opening 9 it is further defined by an outer tube 1 which further defines an outer tube wherein the outer tube is attached to tube stop 6 inside of the box 3. More particular, the outer tube 1 defines an outer lip attached to tube stop 6 inside of box 3. As shown in FIGS. 2 through 5, the outer tube 1 houses a inner tube 2 within the box 3 wherein the inner tube 2 attaches to a top portion of the bag 4 for housing the bag 4 within the box 3.

Referring once again to FIGS. 2-5, the bag 4 of the urine collection device houses and absorbent polymer pouch 5 for absorbing and solidifying urine 7. Additionally, the bag 4 houses and contains an essential oil for masking the smell of urine. And in a preferred embodiment may have a lemon scent smell or any of the essential oil smells used in aromatherapy.

Turning once again to FIGS. 2 through 5, the box 3 of the urine collection device may be a rectangular box 3 which is two inches in width and length and four inches in height. Similarly, the box 3 of the urine collection device may be a square box 3 which is four inches in width, length and height. Lastly, by way of example but not of limitation, the box 3 of the urine collection device may be manufactured from a compostable material.

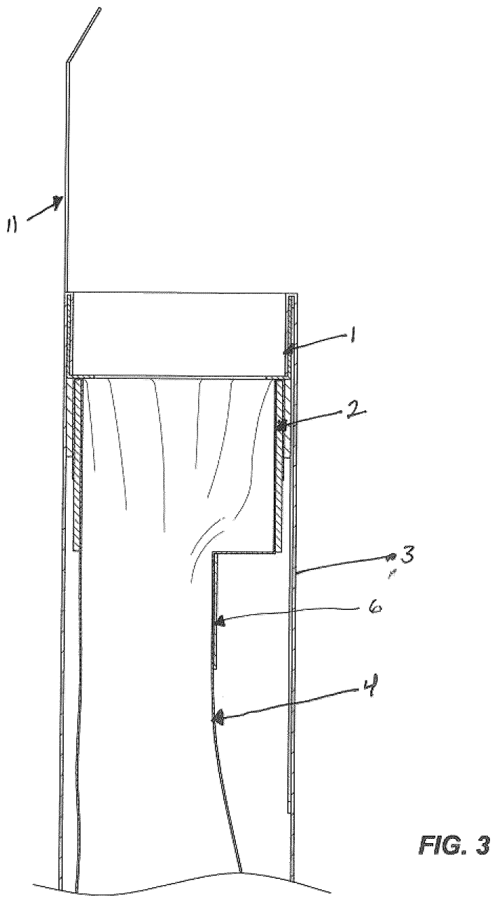

Referring to FIGS. 3 through 5, the portable method for collecting urine, comprises the steps of deploying a bag 4 housed within box 3 defining a front, back, bottom flap 11, a top flap 10 and at least two side edges. The portable method for collecting urine deploys the bag 4 by opening the top flap 10 and opening the bottom flap 11 wherein the bottom of the bag 4 is pulled down for further defining a liquid urine 7 collection area.

In summary, the portable method for collecting urine comprises the steps of defining a top opening 9 housed at a top of a bag 4 for guiding a penis 8 for urination into the bag 4. The portable method for collecting urine receives a user's urine 7 at the absorbant pouch 5 of the bag 4 which houses a polymer material for absorbing and solidifying the urine 7. The portable method for receiving the urine 7 at the absorbing pouch 5 of the bag 4 additionally houses an essential oil for masking the smells of the urine 7 where by way of example but not of limitation may be lemon scented or any scent associated with aromatherapy scents. It should be understood that the portable method includes defining a outer tube 1 housed at the top of the bag 4 which further houses a inner tube 2 for attaching the bag 4 within the box 3 for ready expansion of the bottom of the bag 4.

Lastly, the portable method for collecting includes manufacturing the box, bag, glue and all components from a compostable material. The portable method for collecting urine defines the box 3 as a rectangular box which by way of example but not of limitation may be two inches in width and length and four inches in height. The portable method for collecting urine may also define the box 3 as a square box 3 which by way of example but not of limitation may be four inches in width, length and height. The portable method for collecting urine defines the top outer tube 1 having a diameter of two and a quarter inch housed at the top of the bag 4 for guiding a penis 7 for urination the said bag 4.

No limitation with regard to the described aspects or embodiments of the present invention is intended. Many modifications to the depicted embodiments may be made without departing from the spirit and scope of the present invention. Accordingly, the foregoing description is intended to be illustrative rather than restrictive. The invention described herein is defined by the appended claims and all changes to the invention that fall within the meaning and the range of equivalency of the claims are embraced within their scope.

What is claimed is:

1. A urine collection device, comprising:
A portable bag housed in a box like structure, wherein said bag is housed in a compact condition inside said box, wherein said box comprises a front, back, bottom, top and at least two side edges, with a bag opening formed at the opposite end of the bottom of said bag.

2. The urine collection device according to claim 1 wherein said bag opening is defined by an outer and inner tube further defining an outer lip wherein said outer lip is attached to an inside of said box;
Said outer tube housing a smaller tube within said box wherein said inner tube attaches to a top portion of said bag for housing said bag within said box.

3. The urine collection device according to claim 1 wherein said bag houses and contains a polymer material for solidifying urine.

4. The urine collection device according to claim 1 wherein said bag houses and contains an essential oil for masking the smell of urine.

5. The urine collection device according to claim 1 wherein said box is a rectangular box which is two inches in width and length and four inches in height.

6. The urine collection device according to claim 1 wherein said box is a square box which is four inches in width, length and height.

7. The urine collection device according to claim 1 wherein said bag, box, glue, and all components are manufactured from a compostable material.

8. The urine collection device according to claim 1 wherein said essential oil is lemon scented, or any other essential oils

9. A portable method for collecting urine, comprising the steps of:
Deploying a bag housed with a box defining a front, back, bottom flap, a top flap and at least two side edges.

10. The portable method for collecting urine according to claim 10 wherein said deploying of said bag is achieved by opening said top flap and opening said bottom flap wherein said bottom of said bag is pulled down for further defining a liquid urine collection area.

11. The portable method for collecting urine according to claim 10 further comprising the step of defining a top opening housed at a top of said bag guiding a penis for urination into said bag.

12. The portable method for collecting urine according to claim 10 wherein receiving said urine at said bottom of said bag into a polymer material which solidifies the urine.

13. The portable method for collecting urine according to claim 10 wherein receiving said urine at said bottom of said bag into an essential oil for masking smells of the urine.

14. The portable method for collecting urine according to claim 10 wherein the step of masking urine smells is lemon scented, or any other essential oil

15. The portable method for collecting urine according to claim 10 further comprising the step of defining a outer tube housed at a top of said bag which further houses a smaller tube for attaching said bag within said box for ready expansion of said bottom of said bag.

16. The portable method for collecting urine according to claim 10 further comprising the step of defining a outer tube having a diameter of two and a quarter inch housed at a top of said bag for guiding a penis for urination into said bag.
